(19) United States
(12) Patent Application Publication
(10) Pub. No.: US 2020/0393446 A1
(43) Pub. Date: Dec. 17, 2020

(54) BIOLUMINESCENT SCREENING TEST FOR DETECTING CELL CYTOLYSIS

(71) Applicants: Centre National de la Recherche Scientifique (CNRS), Paris (FR); Universite de Paris, Paris (FR)

(72) Inventors: Simon Hayek, Cachan (FR); Nassima Bekaddour Benatia, Angers (FR); Pierre- Olivier Vidalain, Puteaux (FR); Jean-Philippe Herbeuval, Paris (FR)

(73) Assignees: Centre National de la Recherche Scientifique (CNRS), Paris (FR); Universite de Paris, Paris (FR)

(21) Appl. No.: 16/770,192

(22) PCT Filed: Dec. 7, 2018
(86) PCT No.: PCT/EP2018/084024

§ 371 Date: Jun. 5, 2020

(30) Foreign Application Priority Data
Dec. 8, 2017 (EP) 17306734.9

(51) Int. Cl. G01N 33/50; G01N 21/76

(52) U.S. Cl. CPC G01N 33/5014; G01N 21/763; G01N 33/5047

(57) ABSTRACT

The present invention relates to an in vitro method for testing the cytotoxicity of a candidate compound for living target cells, said method relying on the detection of the massive release of a cytosolic luminescent enzyme in the supernatant of the cells once they have been voluntarily permeabilized. The detection of this release is rapid and reproducible. More precisely, the method of the invention comprises the steps: a) contacting, with a candidate compound, living target cells that constitutively express in their cytosol a luminescent enzyme, b) isolating the supernatant of the coculture of step a) in a second recipient, c) adding in said second recipient the substrate of said luminescent enzyme, and d) measuring the luminescence emitted in said second recipient. The cytotoxicity of said candidate compound is then proportional to the increase in luminescence measured in step d). This method can be advantageously automated so as to assess the cytotoxicity of candidate compounds with high throughput screening devices.

### SUMMARY OF THE INVENTION

The present invention relates to an in vitro method for testing the cytotoxicity of a candidate compound for living target cells, said method relying on the detection of the massive release of a cytosolic luminescent enzyme in the supernatant of the cells once they have been voluntarily permeabilized. The detection of this release is rapid and reproducible.

More precisely, the method of the invention comprises the steps:

a) contacting, with a candidate compound, living target cells that constitutively express in their cytosol a luminescent enzyme,

b) isolating the supernatant of the coculture of step a) in a second recipient,

c) adding in said second recipient the substrate of said luminescent enzyme, and

d) measuring the luminescence emitted in said second recipient.

The cytotoxicity of said candidate compound is then proportional to the increase in luminescence measured in step d).

This method can be advantageously automated so as to assess the cytotoxicity of candidate compounds with high throughput screening devices.

### BACKGROUND OF THE INVENTION

Methods to determine cell viability or cytotoxicity in response to exposure to a given test agent are key to pharmaceutical and environmental testing, pesticide and herbicide testing, drug discovery, etc. In short, to determine whether a given chemical agent presents a real or potential risk when exposed to a given cell type requires a method that reliably, rapidly, precisely, and accurately measures cell toxicity and/or viability after exposure to the test agent.

Cellular cytotoxicity plays an important role also in the immune response. There are two main types of cytotoxic cells: natural killer (NK) cells and cytotoxic T lymphocytes (CTLs). NK cells interact with other cells via an inhibitory receptor and/or an activating receptor, while CTLs utilize the T-cell receptor to recognize antigenic peptides bound to major histocompatibility complex molecules. Both induce target cell lysis through perforin and granzyme release, while CTLs can also induce apoptosis through death ligands such as Fas-Fas ligand interactions. Other immune cells, such as monocytes and dendritic cells, can also express death ligands or cytotoxic substances (i.e. superoxide ions; hydrogen peroxide) to lyse target cells.

To measure the cytotoxic activity of such effector cells in vitro, the chromium release cytotoxicity assay has widely been used and is still the gold standard. However, this assay is limited to a single time point readout only, as the same sample cannot be measured at more than one time point. Moreover, many institutions encourage researchers to limit the time spent in the use of the exposure to radioactive materials. As ⁵¹Cr is radioactive, it is harmful to the researcher's health and it requires special radioactive training. Finally, the cost of safe disposal of radioactive waste must be considered and measuring ⁵¹Cr requires a gamma counter, which is an expensive instrument. Thus, safer alternative methods to measure the cytotoxicity of cells are urgently needed.

A number of alternative methods have been developed that avoid the use of radioactive reagents. Non-radioactive chromium can be used to label target cells and its release from lysed cells can be measured by flameless atomic absorption spectroscopy (FAAS). Correlation of data from this method and the radioactive chromium-release assay is high.

However, FAAS takes a long time to measure chromium-release, as a single sample takes up to 2 minutes to read, making the processing of large numbers of samples problematic. As an alternative approach to chromium release, flow cytometric assays (FACS) that use fluorescent dyes such as carboxyfluorescein succinimidyl ester (CFSE), PKH-2, and PKH-26, which are lipophilic and integrate into the cell membrane, have been developed. These dyes have been used for multiple color analysis via flow cytometry to measure cytotoxicity by human and mouse effector cells (Nakagawa Y. et al, Biomed. Res. 2011). Comparisons between these two methods have shown that the flow cytometric methods could be more sensitive than the chromium-release assay. Yet, these latter methods require a complex apparatus (FACS), qualified users and this kind of analysis is time-consuming.

Other high throughput assays for measuring NK activity have been described in the literature, but all have drawbacks. A test proposed by Lee et al. (Biochem. Biophys. Res. Commun. 2014) measured by ELISA the IFN-γ present in the supernatants of human Peripheral Blood Mononuclear Cells (PBMCs), what reflects the activation of NK. However, this is not a direct measure of the PBMC related cytolytic activity, as IFN-γ can be secreted by cells other than NK cells. In addition, ELISA tests are time consuming and expensive. Other tests consist in co-cultivating PBMCs or NK cells with adherent targets. After incubation, the effector cells are eliminated as well as the targets that have been lysed, and it is then possible to quantify the number of cells still alive in each well by different methods (MTT, alamar Blue®, calcein-AM, etc.) (Wahlberg B J. Et al, J. immunol. Methods 2001). However, these tests can only be used with adherent target cells, and it is not always the case (e.g. K562 cells are not adherent). In addition, they rely on a quantification of cells still alive in the culture wells rather than on a direct quantification of the cell lysis.

Other approaches use the bioluminescence of luciferase-transduced cell lines. Since bioluminescence is ATP-dependent, a dying cell will stop emitting bioluminescence once its remaining intracellular ATP has been used up. Thus, by using stably transduced target cell lines expressing luciferase, NK and CTL cellular cytotoxicity can be detected as a decrease in bioluminescence (Karimi M A et al, PLOS ONE 2014). However, the dynamic range of this test is low (ratio signal max/min signal=3.2 in K562 cells).

Other cell viability tests using the arrest of synthesis of ATP and its degradation in dying cells are used in the art. The CellTiter-Glo® assay (Promega) is one of them. This assay is based on quantitation of the ATP in a culture, which signals the presence of metabolically active cells. ATP presence is detected by means of a thermostable recombinant firefly luciferase. Yet, the cell viability should be assessed at least 30 minutes after cell lysis, which is a delay required for ATP from dead cells to be degraded. Also, the cellular ATP content may vary depending on the metabolic status of the cells, and this may affect the relationship between cell number and luminescence. For example, anchorage-dependent cells that undergo contact inhibition at high densities may show a change in ATP content per cell at high densities, resulting in a nonlinear relationship between cell number and luminescence. Also, factors that affect the cytoplasmic volume or physiology of cells will affect ATP content. For example, oxygen depletion is one factor known to cause a rapid decrease in ATP. Thus, a number of parameters may affect the level of ATP independently of the viability status of the cell, decreasing the sensitivity of the tests. Finally, it can be challenging to detect cell death events in a global population of dividing cells. Indeed, cell division can compensate for cell death events, and it can be difficult to distinguish cytostatic reagents from those inducing some limited cell death without affecting proliferation.

Other viability tests use the arrest of synthesis of a luminescent or fluorescent enzyme. Yet, in these tests, prolonged incubation steps are required to enable the cells' machinery to convert these indicator molecules into a detectable signal. Also, as for ATP, a number of parameters may affect the level of these marker independently of the viability status of the cell, decreasing the sensitivity of the tests.

For all these reasons, indirect measurement is thought to be time-consuming, poorly sensitive and not quantitative.

In this context, there remains a long-felt and unmet need for a cytotoxicity test that measures cell death (rather than viability), in a rapid and non-toxic manner.

Ideally, this test should be efficient on any target cells, adherent or not, that have been subjected to a detrimental experimental condition (e.g., a mechanical stress, a chemical compound, proteins that assemble to form pores into membranes, a viral infection or effector cells).

### DETAILED DESCRIPTION OF THE INVENTION

Non-viable cells that have lost membrane integrity leak cytoplasmic components into the surrounding medium. Cell death thus can be measured by monitoring the concentration of released cellular components in the surrounding medium. One such method is described in Corey et al. (1997) J. Immunol. Meth. 207:43-51.

The loss of integrity of the cell membrane and its permeabilization state is considered as a direct indicator of cell viability and/or cytolysis.

In this context, the present inventors now propose a method to directly detect the loss of integrity of a target cell membrane and its permeabilization state. They have developed two different cell lines that constitutively express a bioluminescent enzyme in their cytosol. When these recombinant cells are alive, the bioluminescent enzyme is sequestered in the cytosol, and the luminescence in the supernatant is limited. The inventors have shown that, when these recombinant cells are subjected to detrimental experimental conditions (e.g., put in contact with effector cells such as NK cells), there is a massive release of the cytosolic bioluminescent enzyme in the supernatant of the cell culture. Said release is detected with a luminometer, whose acquisition time is very short. Therefore, the lysis efficiency will be detected immediately after the substrate is added to the sample.

Importantly, the duration of the test of the invention only depends on the lysis efficiency of the detrimental experimental conditions that are applied to the target cells (the more efficient they are, the quicker the test is). If the detrimental experimental conditions have high lysis efficiency, the output of the cytolytic assay of the invention will be almost instantaneous. The method of the invention is therefore more rapid than most of the viability assays existing in the prior art (e.g., FACS-based).

In general, tests assessing a biomarker's positive increase are known to be more reliable than those based on a biomarker's decrease. As a matter of fact, said decrease can be due to reasons unrelated to the tested event (said reasons can be the degradation of the biomarker in the supernatant, unappropriate concentrations, etc.).

Here, the present application proposes a positive marker of membrane cell permeabilization that depends on the increase of a luminescent signal in the culture supernatant once the cells are subjected to detrimental experimental conditions. The increase of said cytosolic signal is solely and directly controlled by the tested event (e.g., a mechanical stress, contact with a chemical compound or with effector cells, etc.).

The method of the invention is therefore more reliable than those of the prior art.

The method of the invention requires the use of a luminometer, which is far easier to handle than other devices used in the art (flow cytometer and radioactive measurement device for example).

The method of the invention is therefore easier to practice than those available in the art.

Any transfectable cells can be used as target cells (not only adherent cells).

The method of the invention has therefore a broader scope of usefulness than those of the prior art.

The present application relates to an in vitro method for testing the cytotoxicity of experimental detrimental conditions on living target cells, said method comprising at least the following steps:

a) subjecting to experimental detrimental conditions living target cells that constitutively express a luminescent enzyme in their cytosol,

b) isolating the supernatant of the culture of step a) in a separate recipient,

c) adding a define amount of the substrate of said luminescent enzyme in said separate recipient, and

d) measuring the luminescence emitted in said separate recipient,

wherein the cytotoxicity of said experimental detrimental conditions is proportional to the increase in luminescence measured in step d).

As used herein, the term “cytotoxicity” refers to any type of injury that may affect the cellular membrane, leading to its permeabilization or disruption. Partial permeabilization is also testable by the present invention. In fact, with the method of the invention, it is possible to detect cell membrane permeabilization leading to the formation of holes having a diameter of at least 4 nm, for example comprised between 4 nm and 100 nm.

A number of chemical compounds and experimental conditions are known to destabilize the cellular membrane and make the cytoplasmic proteins leak into the surrounding medium. They are for example proteins, peptides, detergents, chaotropic agents, and lytic viruses. Mechanical disruption of membrane can also be carried out by repeated freezing and thawing, sonication, pressure, or filtration. Membrane permeabilization occurs at a speed and doses that depend on the nature of the cells (bacteria, tumoral cells, etc.). Finally, cell death by necrosis, pyroptosis or apoptosis leads to membrane destabilization. Therefore, the duration of step a) varies according to the nature of the lysis stimulus (mechanical/cellular/chemical). The appliance of the experimental detrimental conditions can therefore last in step a) for a variable given time.

When non-denaturing detergents such as Triton X-100, Igepal CA-630, Tween 20 or Brij 35 are used at high concentrations (0.5-5%) cytolysis can be observed on bacteria or human cells rather quickly. In this case, the “given time” for step a) can be comprised between 5 seconds to 5 minutes.

When cytotoxic cells such as NK cells put in contact with tumoral cell lines, the cytolysis can be observed after one hour (preferably after two hours). Therefore, in this case, the “given time” for step a) is comprised between one hour and 6 hours, more preferably comprised between two hours and 5 hours, even more preferably is of four hours.

Without being limited to these propositions, “experimental detrimental conditions” as meant in the present application encompass the following exemplary situations:
to subject the tested cells to an environmental stress such as repeated freezing and thawing, sonication, pressure, filtration, high or low oxygen level, low or high level of radiations, low or high level of humidity, low or high temperature, the presence of a gas,
to put the tested cells in contact with a chemical compound such as a detergent (preferably a non-ionic or a zwitterionic detergent), an antimicrobial agent, a cytotoxic drug, a chemotherapeutic agent, an antibiotic,
to put the tested cells in contact with a population of effector cells (for example NK cells, cytotoxic T lymphocytes, monocytes, macrophages, dendritic cells, or virus-infected cells), optionally along with a modulator of said effector cells,
to put the tested cells in contact with microorganisms (virus or bacteria),
to put the tested cells in contact with cytolytic proteins (such as perforins or bacterial exotoxins) or peptides (such as frog temporine), death ligands (FasL, TRAIL, TNFα, etc.) or a complex of cytolytic proteins (for example the complex C9 of the complement, dermaseptins, membrane-damaging exotoxins from bacteria such as Panton-Valentine leukocidin).

Any living transfectable target cells can be used in this method, provided that they can be efficiently transformed, transfected or transduced with a recombinant vector. In particular, they can be vertebrate or “non-vertebrate” (or invertebrate) cells, plant cells, yeast cells, prokaryote cells, eukaryote cells, archea, bacteria, etc.

Non-vertebrate (also known as invertebrate) comprises different phyla, the most famous being the Insect, Arachnida, Crustacea, Mollusca, Annelida, Cirripedia, Radiata, Coelenterata and Infusoria. They are now classified into over 30 phyla, from simple organisms such as sea sponges and flatworms to complex animals such as arthropods and molluscs. In the context of the invention, non-vertebrate cells that can be used as target cells are preferably insect cells, such as Drosophila or Mosquito cells, more preferably Drosophila S2 cells.

Examples of cells derived from vertebrate organisms that are useful as target cell lines in the context of the invention include non-human embryonic stem cells or derivative thereof, for example avian EBX cells; monkey kidney CVI line transformed by SV40 sequences (COS-7, ATCC CRL 1651); a human embryonic kidney line (293); baby hamster kidney cells (BHK, ATCC CCL 10); Chinese hamster ovary cells (CHO); mouse sertoli cells [TM4]; monkey kidney cells (CVI, ATCC CCL 70); African green monkey kidney cells (VERO-76, ATCC CRL-1587); human cervical carcinoma cells (HeLa, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor cells (MMT 060562, ATCC CCL51); rat hepatoma cells [HTC, MI.5]; YB2/O (ATCC n° CRL1662); NIH3T3; HEK 293, K562 and TRI cells. In the context of the invention, vertebrate cells are preferably HEK 293 or K562 cells, or derivatives thereof.

Plant cells which can be used as target cells in the context of the invention are the tobacco cultivars Bright Yellow 2 (BY2) and Nicotiana Tabaccum 1 (NT-1).

Yeast cells which can be used as target cells in the context of the invention are: Saccharomyces cerevisiae, Schizosaccharomyces pombe, and Hansenula polymorpha, as well as methylotropic yeasts like Pichia pastoris and Pichia methanolica.

Prokaryote cells which can be used as target cells in the context of the invention are typically E. Coli bacteria or Bacillus Subtilis bacteria.

The medium in which the cells are grown or held does not limit the functionality of the invention. For microbial cultures, suitable media include, without limitation, Mueller-Hinton Broth and trypticase soy broth. For mammalian cell cultures, suitable media include, without limitation, RPMI 1640, RPMI 1640 plus fetal bovine serum, and Dulbecco's Modified Eagle Medium, Hanks' Balanced Salt Solution, Phosphate Buffered Solution.

In a preliminary step, these target cells have been transfected with a vector carrying a nucleotide sequence encoding a luminescent enzyme, so that stable expression of this enzyme in their cytosol is allowed.

The term “vector” herein means the vehicle by which a DNA or RNA sequence of a foreign gene can be introduced into a host cell so as to transform it and promote expression of the introduced sequence. Vectors may include for example, plasmids, phages, and viruses and are discussed in greater detail below. Indeed, any type of plasmid, cosmid, YAC or viral vector may be used to prepare a recombinant nucleic acid construct which can be introduced to a host cell where expression of the protein of interest is desired. Alternatively, wherein expression of the protein of interest in a particular type of host cell is desired, viral vectors that selectively infect the desired cell type or tissue type can be used.

Viral vectors are for example lentiviruses, retroviruses, herpes viruses, adenoviruses, adeno-associated viruses, vaccinia virus, baculovirus, and other recombinant viruses with desirable cellular tropism. Methods for constructing and using viral vectors are known in the art.

For transfecting vertebrate cells, lentiviral, AAV, baculoviral and adenoviral vectors are preferred. The vectors suited for expression in mammalian host cells can also be of non viral (e.g. plasmid DNA) origin. Suitable plasmid vectors include, without limitation, pREP4, pCEP4 (Invitrogene), pCI (Promega), pCDM8 and pMT2PC, pLVX (Clontech), pVAX and pgWiz.

For prokaryote cells, plasmid, bacteriophage and cosmid vectors are preferred. Suitable vectors for use in prokaryote systems include without limitation pBR322 (Gibco BRL), pUC (Gibco BRL), pBluescript (Stratagene), p Poly, pTrc, pET 11d, pIN, and pGEX vectors.

Expression of recombinant proteins in yeast cells can be done using three types of vectors: integration vectors (YIp), episomal plasmids (YEp), and centromeric plasmids (YCp). Suitable vectors for expression in yeast (e.g. S. cerevisiae) include, but are not limited to pYepSec1, pMFa, pJRY88, pYES2 (Invitrogen Corporation, San Diego, Calif.) and pTEF-MF (Dualsystems Biotech Product code: P03303).

The target cells used in the present application should constitutively express the luminescent enzyme in their cytosol. The skilled person knows how to generate such cells. They know in particular which vectors have to be used to address the recombinant enzymes to the cytosol of the transfected cells and what promoter they have to associate to said luminescent enzyme for triggering a constitutive expression of the enzyme.

Promoters suitable for constitutive expression in mammalian cells include the cytomegalovirus (CMV) immediate early promoter (CMV IE promoter), the adenovirus major late promoter, the phosphoglycero kinase (PGK) promoter, and the thymidine kinase (TK) promoter of herpes simplex virus (HSV)-1.

Preferably, the promoter used in the vector transfecting the said target cells is the CMV IE promoter.

A range of yeast promoters is available for protein expression in yeast host cells. Some like GAPDH are constitutive in expression. Other promoters suitable for expression in yeast include the TEF, PGK, MF alpha, CYC-1, GAL-1, GAL4, GAL10, PHO5, glyceraldehyde-3-phosphate dehydrogenase (GAP or GAPDH), and alcohol dehydrogenase (ADH) promoters.

For use in plant cells, the most commonly used promoter is the cauliflower mosaic virus (CaMV)35S promoter or its enhanced version, but a number of alternative promoter can be used.

Promoters suitable for expression in E. coli host cell include, but are not limited to, the bacteriophage lamba pL promoter, the lac, and TRP promoters.

The transfection of the target cells with the designed polynucleotides can be performed by any classical method in the art, for example by transfection, infection, or electroporation. Alternatively, the vectors can be introduced in the target cells by lipofection (as naked DNA), or with other transfection facilitating agents (peptides, polymers, etc.). Other molecules are also useful for facilitating the transfection of a nucleic acid, such as cationic oligopeptides (see WO 95/21931), peptides derived from DNA binding proteins (see WO 96/25508), or a cationic polymer (see WO 95/21931). It is also possible to introduce the vector in the cells as a naked DNA plasmid. Naked DNA vectors can be introduced into the desired host cells by methods known in the art, such as electroporation, microinjection, cell fusion, DEAE dextran, calcium phosphate precipitation, use of a gene gun, or use of a DNA vector transporter.

Once the vectors carrying the nucleotide sequence of the luminescent enzyme have been introduced in the target cells e.g., by a means defined above, the expression of said luminescent enzyme is allowed. This means that the stimulus of the regulatory sequences that are present in the vector and all the required components are present in a sufficient amount for the translation of the polynucleotide to occur.

The expression level of the luminescent enzyme in the cytosol of the target cells can be assessed by different means that are well known in the art (typically by luminometry).

Any type of luminescent enzyme can be used in the context of the invention, provided that it is stable in time, has a small size (so as to be easily released from the cytosol to the surrounding medium once permeabilization of the membrane occurs), and that it is sufficiently luminescent to be detected by conventional luminometers even in small amounts.

As mentioned above, the present application relates to the use of cells that constitutively express in their cytosol a luminescent enzyme, for detecting the cytotoxicity of a candidate compound or experimental conditions. Accordingly, the luminescent enzyme should not be operatively connected to a peptide secretion signal.

Luminescence refers to the light output of a luciferase polypeptide under appropriate conditions, e.g., in the presence of a suitable substrate (such as coelenterazine or furimazine, see below). In the present invention, the luminescence reaction will take place in the extracellular medium of the target cells. This medium may contain proteases or other toxic components (detergents for example) which can affect the enzyme functionality. That's why it is important to use an enzyme that is structurally stable in such medium. It is also important to use an enzyme whose luminescent signal (in the presence of its substrate) is strong in such medium.

Accordingly, the luminescent enzyme used in the method of the invention should have an appropriate protein stability, i.e. a high thermal stability (at room temperature, or at more elevated temperatures) and/or a high chemical stability (in the presence of denaturants such as proteases or detergents). This enhanced stability is required especially when the exposure of the cells to the tested experimental conditions results in release of the cytoplasmic component several hours before the substrate is added. In particular, the luminescent enzyme should be able to remain unaffected when stored in a biological media including cellular lysates (and therefore proteases) for 2 days at 37° C.

Said luminescent enzyme is typically more stable than the firefly or renilla luciferase used in the art. Of note, the half-life of the NanoLuc enzyme used in the experimental part of the application is of 7.7 days when stored in a lysate of HEK293 cells, whereas, in the same lysates, the half-life of the firefly luciferase is of 7.3 minutes and the half-life of the renilla luciferase is of 99 minutes (Hall M P et al, ACS Chem Biol 2012, 7, 1848-1857).

The luminescent enzyme used in the method of the invention should also have an appropriate luminescence, i.e., a high luminescence signal when its appropriate substrate is added. Preferably, said signal remains almost unaffected long after the substrate is added. In particular, the signal should be detectable during at least ten minutes in a biological media including cellular lysates, even if small amount of the luminescent enzyme is present. The luminescent activity of said luminescent enzyme is typically stronger than that of the firefly or renilla luciferase used in the art. For example, the luminescence intensity of the NanoLuc enzyme used in the experimental part of the application is 150 folds stronger than the luminescence intensity of the firefly luciferase or of the renilla luciferase (Hall M P et al, ACS Chem Biol 2012, 7, 1848-1857).

Eventually, the luminescent enzyme used in the method of the invention has a typical size comprised between 10 and 100 kDa, preferably between 10 and 60 kDa, more preferably between 10 and 40 kDa, even more preferably between 10 kDa and 20 kDa, so as to be easily released from the cytosol to the surrounding medium once permeabilization of the membrane occurs.

In the method of the invention, it is possible to use natural or synthetic luciferases. Natural luciferase includes the shrimp Oplophorus luciferase, the sea pansy Renilla luciferase, the deep sea medusa Periphylla luciferase, the planktonic copepods Gaussia luciferase, the planktonic copepods Metridia luciferase, the crustacean ostracods Conchoecia luciferase and the crustacean ostracods Vargula luciferase, the fish Myctophina luciferase, the squid Watasenia scintillans luciferase, the squid Symplectoteutis oualaniensis luciferase, the mollusk P. dactylus luciferase, etc. Synthetic luciferases having an enhanced stability or signal stability or brightness as compared with these natural enzymes can be designed and generated by molecular means well known in the art. One such example is disclosed in EP 3 181 687, which discloses a number of recombinant enzymes having increased luminescence emission relative to the corresponding wild-type luciferase (in this case the shrimp Oplophorus luciferase, the Heterocarpus luciferase, the Systellapis luciferase, the Acanthephyra luciferase, the Lingulodinium polyedrum luciferase, and the Pyrocystis lunula luciferase).

A number of appropriate substrates for luciferase enzymes have been described in the art. They are for example D-luciferine, coelenterazine, coelenteramide bisulfate, coelenterazine bisulfate, dehydrocoelenterazine, furimazine or any other bioluminescent imidazole [1,2-a]pyrazine derivatives (see Coutant E P & Janin Y L, Chem. Eur. J., 2015). In the context of the invention, it is possible to use any substrate that enables to generate a sustained and stable luminescent signal when associated with the selected luminescent enzyme. The skilled person well knows how to choose the appropriate luciferase substrates for enhancing and/or stabilizing the luminescent signal emitted by the luciferase enzyme (see Coutant E P & Janin Y L, Chem. Eur. J., 2015). Preferably, the luminescent enzyme used in the method of the invention uses its substrate in an ATP-independent reaction.

In a particularly preferred embodiment, the method of the invention uses the recombinant portion of Oplophorus luciferase, made out of its 19 kDa catalytic component, which is using furimazine or bisdeoxycoelenterazine as well as 6h-f-coelenterazine as substrates, in an ATP-independent reaction. One more particularly preferred luminescent enzyme is the NanoLuc enzyme commercialized by Promega. Vectors containing the nucleotides encoding said enzyme can be provided by Promega under the reference number pNL1.1.CMV[Nluc/CMV].

Step b) of the method of the invention requires isolating the supernatant of the culture of step a) in a separate recipient. Said recipient is preferably a MW 96 well, a microtiter plate, a test tube, or any suitable vessel.

Said isolation can be performed by any conventional means, such as by transferring a define volume of the supernatant in said recipient or by filtration.

The amount of the transferred supernatant can be comprised between 1 μL and 1 mL. Preferably, it is comprised between 1 μL and 500 μL. More preferably, it is of about 20 μL.

Once the supernatant has been isolated from the target cells, a non-limiting amount of the substrate of the selected luminescent enzyme is added in said supernatant. Said amount is determined by the skilled person by conventional means. When furimazine is associated to the NanoLuc enzyme, the amount of furimazine is of about 10 to 50 μM.

Once the substrate is added in the supernatant contained in the separate recipient, the luminescence emitted in said separate recipient is measured by luminometer. This is step d) of the method of the invention. Importantly, this measure can be done immediately after adding the substrate to the supernatant of the cells. In other words, data gathering can begin promptly after adding the substrate in the separate recipient. In fact, the signal generated by the method may be measured for anywhere from a matter of seconds to one minute, to ten minutes, to thirty minutes or even longer after the substrate is added. In a preferred embodiment, the signal is measured following a period of time generally on the order of about 0 second, 2 seconds, 5 seconds, 10 seconds, 15 seconds, 20 seconds, 30 seconds, 45 seconds, one minute, 2 minutes, 5 minutes, or 10 minutes after the substrate is added to the recipient.

The light output or luminescence may be reported as the average over time, the sum of the signal over a period of time, or as the peak output.

In a preferred embodiment, the measuring step d) occurs no later than one or two minutes (in case a detergent is tested), three or four hours (in case effector cells are tested) or one to four days (in case a virus is tested) after the living target cells have been exposed to the detrimental experimental conditions.

In another preferred embodiment, the measuring step d) occurs no later than five minutes after the substrate has been added in the supernatant of the cells.

The method described herein is inherently quantitative in that the amount of the released luciferase is proportional to the number of cells lysed. The method, however, can be used both for quantitative testing and qualitative testing. The term “qualitative testing” refers to testing apparatus and methods which produce test results that generally indicate whether an organism or cellular specimen is sensitive or resistant to a particular antibiotic or cytotoxic test agent or other experimental conditions. The relative degree of sensitivity or resistance is not reported in qualitative testing. The term “quantitative testing” refers to testing apparatus and methods which produce test results that provide data on the concentration of the antimicrobial or cytotoxic product that will be sufficient to inhibit growth of the microorganism or other cell type.

In another preferred embodiment, at least one step of the cytotoxicity assay of the invention is automated, so as to provide high scale testing levels. It is indeed possible to provide an automated apparatus that will carry out the transfer of the supernatant, the adding of the substrate, and/or the measurement of the luminescence automatically. The step of contacting living target cells with different compounds or subjecting same to environmental stress as mentioned above can also be done automatically. In a more preferred embodiment, the whole assay of the invention is automated.

Several concentrations (dilutions range) of a cytotoxic compound could for example be tested at once. Also, a number of different target cells could be subjected concomitantly to the same environmental conditions.

A luminometer would be used to detect the release of the luminescent enzyme in the supernatant of the target living cells once they have been put in contact with the tested cytotoxic compound or subjected to other experimental conditions.

Importantly, the cytotoxicity assay of the invention can be used to test the cytolytic activity of candidate compounds that can then be used in therapeutic, cosmetic, veterinary, dermatologic, sanitary, alimentary, or any other technical field.

In another aspect, the present application relates to an automated method for screening concomitantly the cytotoxicity of several candidate compounds in a limited period of time, said method repeating the steps of the cytotoxic assay of the invention as detailed above, for each candidate compound concomitantly.

In another aspect, the present application relates to an automated method for screening concomitantly the cytotoxicity of a candidate compound on a number of different living cells in a limited period of time, said method repeating the steps of the cytotoxic assay of the invention, for each target cells concomitantly.

It is obviously possible to screen concomitantly and automatically the effect of several candidate compounds on several target cells, by combining the two above-mentioned automated methods.

NK (Natural Killer) cells play an essential role in the immune response against viral infections and cancers. These cells have indeed a set of receptors allowing them to detect a cell infected by a virus or that would engage in a process of tumor transformation. The NK cells then secrete a set of factors that will perforate the membrane of the target cells (perforin/granzyme system) or engage proapoptotic receptors (DR4/5, FAS, etc.), and thus induce their death. As such, NK cells are a target of choice in immunotherapy, and it is important to be able to quantify their cytotoxic activity in vitro or ex vivo to evaluate the impact of small therapeutic molecules, antibodies or proteins on this essential component of the innate immune system.

In a preferred embodiment, the present application therefore relates to an in vitro method for testing the cytotoxicity of a population of NK cells on living target cells, said method comprising at least the following steps:

a) contacting living target cells that constitutively express in their cytosol a luminescent enzyme with a population of NK cells for a given time,

b) isolating the supernatant of the coculture of step a) in a separate recipient,

c) adding a define amount of the substrate of said luminescent enzyme in said separate recipient, and

d) measuring the luminescence emitted in said separate recipient,

wherein the cytotoxicity of said population of NK cells is proportional to the increase in luminescence measured in step d).

All the embodiments disclosed above for the cytotoxicity assay of the invention apply mutatis mutandis for this NK cytotoxicity assay. It is also possible to apply a similar method for assessing the cytotoxicity of T CD8+ lymphocytes, of monocytes, of macrophages, or of dendritic cells.

The present inventors disclose in the experimental part below an example of such a test. Without being bound to these particular embodiments, they show that some parameters can be optimized when the NanoLuc enzyme of Promega and furimazine are used as luminescent enzyme/substrate system.

In this embodiment, it is possible to use any source of NK cells, such as human peripheral blood mononuclear cells (PBMC cells), said cells containing only a portion of NK cells. Recovery of PBMC and their treatment to obtain NK cells are well known in the art. They need not to be repeated here. Of note, NK cells from PBMCs do not need to be purified. In the methods of the invention, it is indeed possible to use a population of PBMCs containing 4-8% of NK cells.

The target cells are preferably tumoral cell lines (e.g., K562 or HEK293) that have been transfected with a lentivirus encoding the stable luminescent enzyme described above.

Typically, the ratio between NK cells and target cells can be comprised between about 0.1:1 to about 10:1 (for purified NK), preferably between about 0.5:1 and 5:1 (for purified NK).

As shown in the experimental part below, the present inventors determined the cytolytic activity of these PBMCs/NK cells after 4 hours of coculture with target cells. They were able to assess the lysis of target cells in a 100 of culture wells in less than 5 minutes by measuring luciferase activity in culture supernatants, as opposed to more than one hour with the prior art methods, for example FACS. Moreover, they obtained a better sensitivity than the systems of the prior art (ratio signal max/min signal=20).

This cytotoxic assay offers a superior non-radioactive alternative to the assays of the prior art, with increased signal-to-noise ratio and faster kinetics. It is therefore more robust and quicker to perform.

The present inventors were also able to detect the immunomodulatory effect of two different compounds (one stimulating, one inhibiting) on these effector cells.

It is therefore another purpose of the invention to provide a method for detecting the immunodulatory efficiency of a candidate compound on NK cells, comprising the following steps:

a) Testing the cytotoxicity of the NK cells in the absence of said candidate compound, by means of the testing method described above,

b) testing the cytotoxicity of the NK cells in the presence of said candidate compound, by means of the testing method described above.

If the cytotoxicity measured in step b) is lower than the cytotoxicity measured in step a), then it can be concluded that the candidate compound has an immunosuppressive activity.

If the cytotoxicity measured in step b) is higher than the cytotoxicity measured in step a), then it can be concluded that the candidate compound has an immunostimulatory activity.

Typically, the ratio between NK cells and target cells can be comprised between about 2:1 to about 0.5:1 (for purified NK), for reducing the basis level of the lysis in absence of the tested compound.

A luminometer would be used to detect the release of the luminescent enzyme in the supernatant of the target living cells once they have been contacted with the effector cells in the presence and absence of the tested compound.

The inventors herein provide an example wherein the effect of two immunomodulatory molecules (R848 and Clobenpropit) have been tested successfully on the cytolytic activity of PBMC/NK cells.

This test can also be used to test the immunodulatory efficiency of candidate compounds on other effector cells, such as cytotoxic T lymphocytes cells (also known as CTL or T CD8+), macrophage cells, etc.

In this particular embodiment, the nature of the tested candidate compound is virtually unlimited. It can be, for example, any element, compound, mixture, drug, or putative drug, in any form (such as solid, liquid, or gas) desired to be tested for its functional effects on the effector cells. The method can also be used to test known antimicrobial agents and cytotoxic drugs for the purpose of determining which chemotherapeutic agents would be most effective against a given infection or cell type. The method can also be utilized with unknown or suspected antimicrobial agents and drugs for the purposes of determining their potential activity against a given microorganism or cell type, e.g., high-throughput screening of substances for biological activity as part of drug screening or other activities.

The utility of the assay is manifest in that determining the cytotoxicity of a given compound or composition is a critical piece of information in the drug discovery process. For example, cytotoxicity is desirable when the toxicity is specifically displayed against an identified target cell type, such as a given type of cancer, an infectious, disease-causing microorganism, or a parasitic organism. Cytotoxicity, of course, is undesirable when, for example, a putative new drug is discovered to be cytotoxic to normal cells. The subject invention can be used in both instances to measure the cytotoxicity of a given test agent against a given cell type.

In one embodiment, this test is used to assess the antiviral properties of candidate compounds, by replacing the NK cells by a virus that will infect target cells expressing luciferase. In this case, the limited amount of time is a matter of few hours (for highly cytolytic viruses) or few days (for less virulent viruses), depending on the cytolytic activities of said virus. The skilled person well knows how to adapt the above-mentioned tests to such as purpose.

In another embodiment, this test can be used to assess the anti-microbial properties of candidate compounds, by replacing the NK cells by bacterial cells such as Gram-negative bacteria (e.g., Pseudomonas aeruginosa, Neisseria gonorrhoeae, Chlamydia trachomatis, and Yersinia pestis). In this case, the limited amount of time is a matter of hours to days, depending on the cytolytic activities of said bacteria cells.

The present application also encompasses automated methods for screening concomitantly the modulatory effect of several candidate compounds on the cytotoxicity of these effector cells. These methods repeat the steps of the detecting method exposed above, using the target cells, effector cells and candidate compounds. The modulatory effect of a tested compound is detected when the luminescence increase in the supernatant of the target cells cocultured with the effector cells is affected by the presence of said compound.

Correspondingly, the present application encompasses automated methods for screening concomitantly the modulatory effect of candidate compounds on the cytotoxicity of effector cells on different living cells.

It is obviously possible to screen concomitantly and automatically the modulatory effect of several candidate compounds on the cytolytic activity of several effector cells on several target cells, by combining the methods mentioned above.

The present invention is also drawn to kits that contain reagents and instructions necessary to carry out the above methods.

In a preferred embodiment, these kits include at least one target cell line that constitutively expresses a luminescent enzyme in their cytosol. These cell lines have been detailed above and all of them are within encompassed. The luminescent enzyme is also as defined above.

In another preferred embodiment, these kits include an assay buffer containing the substrate of the luciferase disposed in a separate container. It is preferred that the substrate mixture be present in the form of a lyophilized powder that is then reconstituted with a buffer which contains the components required to practice the method of the present invention. This embodiment is preferred because the lyophilized substrate mix is easier to store and transport. The kit, however, may also be configured so that the necessary components listed above are packaged in the form of a pre-made reagent mix, ready for use.

An optional additional container may hold a lysis solution (for positive control experiments).

These kits should also include instructions for how to carry out the methods of the invention.

### FIGURE LEGENDS

FIG. 1 reveals how the activity of the NanoLuc enzyme is increased in the supernatant of K562 cells expressing NanoLuc constitutively (K562-NanoLuc cells) once these cells were contacted with different cytolytic compositions, such as the NP40 detergent (A), or

Peripheral Blood Mononuclear Cells (PBMC) (B) and (C). In (B), different PBMC/K562-NanoLuc cell ratio have been used, 16 000 K562-NanoLuc cells being used with an increasing number of PBMC. The cells have been co-cultured for 4 hours before the supernatant was collected. “CT−” corresponds to the negative control (K562-NanoLuc cells alone) and “CT+” corresponds to the positive control (K562-NanoLuc expressing cells lyzed with NP40 as in (A). On (C), the same conditions as in (B) have been used with a ratio of 400 000 PBMCs to 16 000 K562-NanoLuc cells, with different time of incubation. The graphs show the NanoLuc activity in the supernatant of these cultures.

FIG. 2 shows the role of NK cells in the cytolytic effect of the PBMCs. K562-NanoLuc cells have been co-cultured for 4 hours with PBMCs, PBMCs depleted in NK cells (w/o NK) and PBMCs depleted in NK that have been supplemented with purified NK cells (w/o NK+NK). 400 000 PBMCs for 16 000 K562-NanoLuc cells have been used (A). Increasing amounts of purified NK cells isolated from peripheral blood have also been contacted with 16 000 K562-NanoLuc cells at different ratio (B). The graphs show the NanoLuc activity in the supernatant of these cultures, after four hours of incubation.

FIG. 3 shows the activity of the NanoLuc enzyme in the supernatant of K562-NanoLuc cells contacted with modulated PBMC effector cells. (A) PBMCs have been incubated for 16 hours with or without R848 as an activator, then K562-NanoLuc cells have been added (200 000 PBMCs/16 000 K562-NanoLuc cells). (B) PBMCs have been incubated for 16 hours with or without Clobenpropit (CB), then K562-NanoLuc cells have been added (400 000 PBMCs/16 000 K562-NanoLuc cells). The graphs show the NanoLuc activity in the supernatant of these cultures, after four hours of incubation.

FIG. 4 shows a scheme of the different steps of the method of the invention.

FIG. 5 shows the cytopathic effects associated to measles virus infection measured by NanoLuc quantification in the supernatants of TwInne cells.

### EXAMPLES

I. Material and Methods

Cell Lines, Peripheral Blood Mononuclear Cells and Culture Conditions

Cells were cultured at 37° C. and 5% CO2 in RPMI-1640 medium (Sigma-Aldrich; R8758) containing 10% fetal calf serum (FCS). Human K562 cells were kindly provided by Dr. T. Walzer (CIRI, Lyon). The reporter cell line STING-37, corresponding to HEK-293 cells stably transfected with the ISRE-luciferase reporter gene, was previously described (PMID:24098125). Blood from healthy blood bank donors was obtained from “Etablissement Francais du Sang” (Convention #07/CABANEL/106; Paris; France). Human peripheral blood mononuclear cells (PBMC) were isolated by density centrifugation with Lymphoprep medium (StemCell Technologies).

Establishment of K562-NanoLuc and twINNE Cell Lines

The NanoLuc gene was introduced in K562 and STING-37 cell lines by transduction with the pLVX-Puro lentiviral vector (Clontech). First, the NanoLuc sequence was cloned in the pLVX-Puro vector using the Gateway cloning system (Thermo Fisher Scientific). Briefly, the pLVX-Puro plasmid was made compatible with the Gateway system by inserting the Gateway cassette C1 at the SmaI site to generate the new destination vector called pLVX-Puro-GW. A pDONR207 plasmid containing the coding sequence for the NanoLuc enzyme (Promega) was used. The NanoLuc sequence was transferred by in vitro LR recombination from pDONR207 to pLVX-Puro-GW vector following manufacturer's recommendations to obtain pLVX-Puro-NanoLuc. This vector was used to produce lentiviral particles by co-transfection with packaging plasmids pVSV-G and pGag-Pol (Naldini & al, Science. 1996 Apr. 12; 272(5259):263-7) into HEK-293T cells. Lentiviral particles were collected two days later and applied to K562 or STING-37 cells for transduction. Cells were treated for 4 weeks in the presence of puromycin to enrich for transduced cells. More than 80 clones were isolated and tested for the expression of NanoLuc. Best expressing clones derived from each K562 and STING-37 cells line were selected, and called K562-NanoLuc and twINNE, respectively.

NK Cell Cytotoxicity Assay

PBMC from healthy donors were incubated overnight in the absence or presence of compounds to be tested. Unless specified otherwise, PBMC were plated in 96-well round-bottom plates at a concentration of 400,000 cells/well in a final volume of 100 μL. After 16 hours, 16.000 K562-NanoLuc or twINNE cells were added to cultures wells (100 μL of cell suspension at 160,000 cells/ml). Total volume in culture wells was 200 μL, and plates were centrifuged briefly for 2 min at 1,200 rpm. Unless specified otherwise, 20 μL of culture supernatants were collected and NanoLuc activity was determined by adding 40 μL of RPMI-1640 and 30 μL of NanoLuc reagent (Promega) in black, flat-bottom, 96½-well plates. Bioluminescence was measured during 0.1 s with a luminometer (Enspire; Perkin Elmer).

Lysis with Measles Virus

Twlnne cells correspond to a clone of human HEK-293 cells stably transfected with a reporter gene for innate immunity (a firefly luciferase sequence controlled by an ISRE promoter) and a second reporter gene encoding the constitutive expression of the NanoLuc luciferase enzyme (obtained by transduction with a pLVX lentivector encoding NanoLuc). TwINNE cells were infected with measles virus (MeV; Schwarz vaccine strain) at indicated “multiplicity of infection” (MOI) that correspond to infectious viral particles per target cells. Cell were incubated for 24, 48 or 72 hours in 96 well plates at an initial concentration of 35.000 cells/well and a final volume of 200 μL. At indicated timepoints, 20 μL of culture supernatants were collected and the NanoLuc activity determined as already detailed above.

II. Results

The present inventors have developed K562 or HEK-293 cell lines that constitutively express the bioluminescent enzyme NanoLuc from a transgene (introduced with a non-replicating lentiviral vector).

NanoLuc is a small reporter protein (of 19 kDa) developed by Promega from a bioluminescent enzyme of the shrimp Oplophorus gracilirostris. It is 150 times brighter than the firefly or renilla luciferases, and display an enhanced stability.

When these K562 or HEK-293 cells are alive, the NanoLuc is sequestered in the cytosol, and the NanoLuc activity in the supernatant is null.

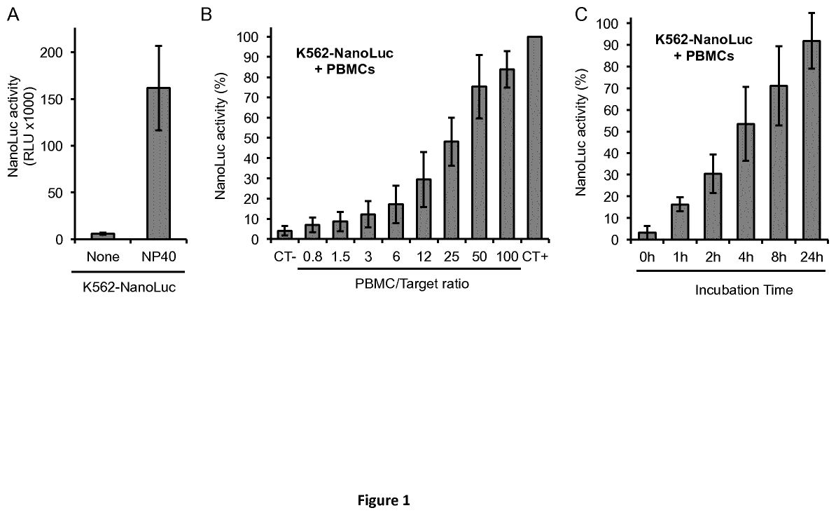

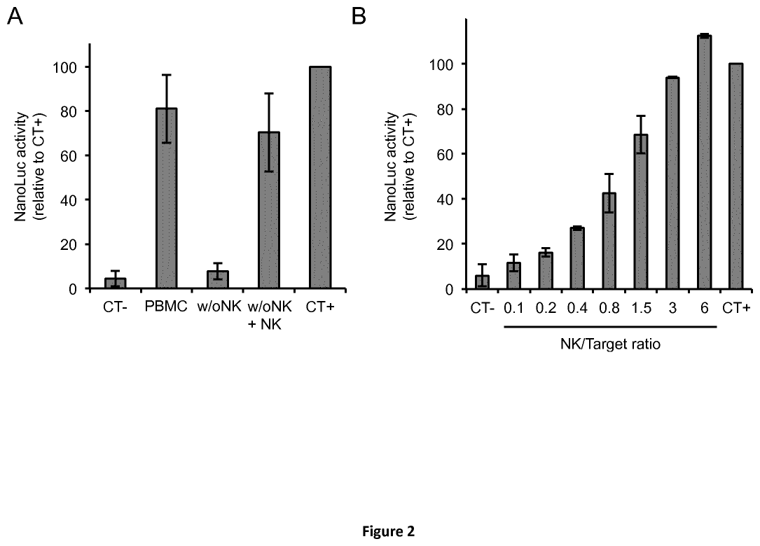

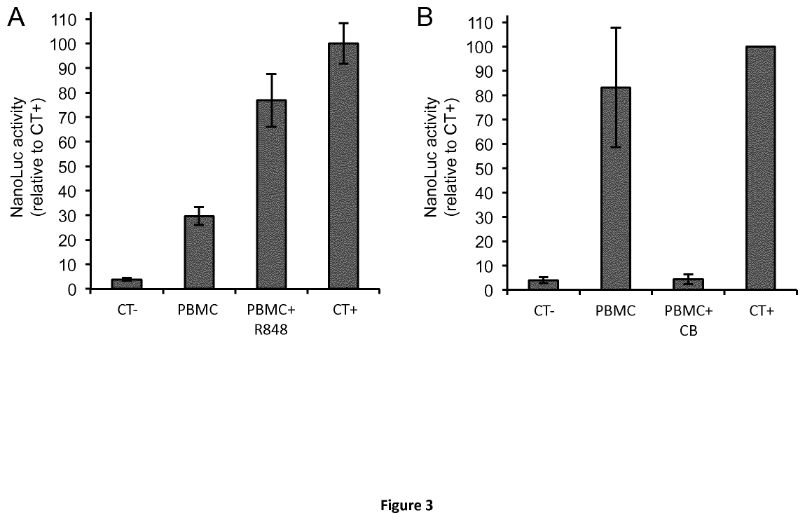

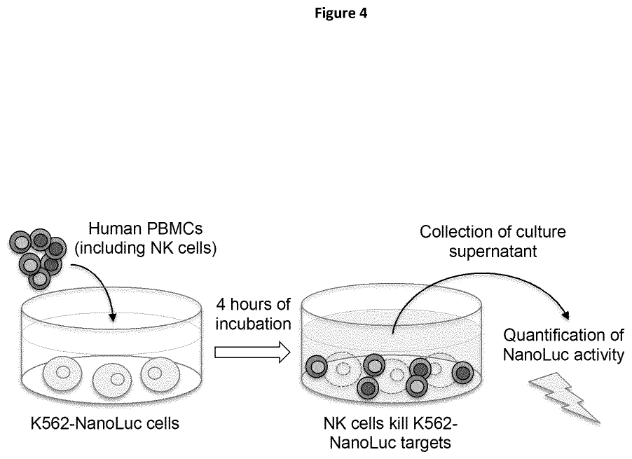

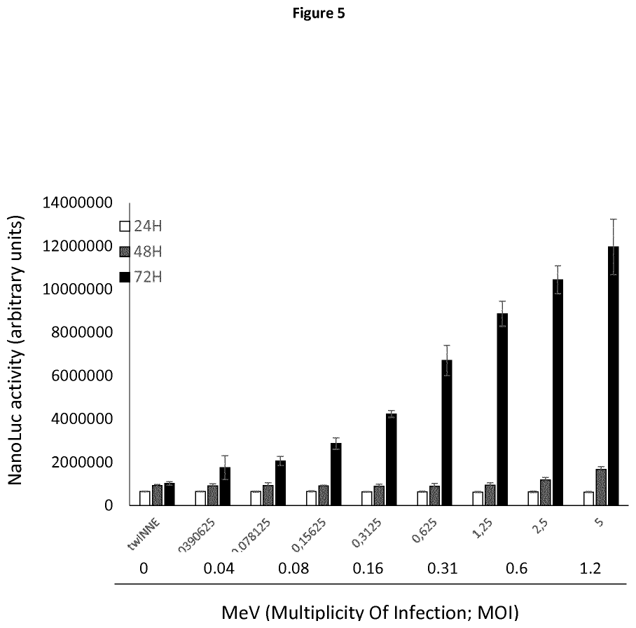

On the other hand, when these NanoLuc expressing lines are incubated in the presence of human peripheral blood mononuclear cells (PBMCs) which contain 1-6% of NK cells, a massive release of NanoLuc is observed in the culture supernatant as evidenced by the increase in enzymatic activity measured by bioluminescence (FIG. 1B).

The inventors have set up the optimal conditions for carrying out this test: 16,000 K562 or HEK-293 cells for 400,000 PBMCs in a total volume of 200 μL of culture medium.

When these conditions are used, a strong signal is observed after only 4 hours of co-culture (FIG. 10).

On the other hand, when PBMCs are depleted in NK cells, the NanoLuc enzyme no longer accumulates in culture supernatants (FIG. 2A). This highlights the role played by NKs in target cytolysis. As expected, it is also possible to perform this test with purified NK cells from peripheral blood (FIG. 2B).

Moreover, the inventors have shown that the test of the invention enables to measure the impact of activating or inhibitory molecules on the cytolytic activity of effector cells such as NK cells. When the PBMCs are incubated in the presence of the compound R848, this synthetic ligand of TLR7 and 8 activates the dendritic cells and other antigen presenting cells (APCs) present within the PBMCs. APCs then activate NK cells through direct interactions and secretion of cytokines. Using the assay of the invention, the inventors were able to demonstrate the increase in the cytotoxic activity of NK cells (FIG. 3A). In this case, only 200,000 PBMCs were used for 16,000 K562-NanoLuc targets (ratio 12.5/1) to reduce the baseline lysis of targets in the absence of R848.

Conversely, when PBMCs are treated with Clobenpropit (CB), a synthetic molecule close to histamine recently identified as a potent immunosuppressant, the cytolytic activity of NK cells is completely inhibited (FIG. 3B). These data show that it is possible to use this test to evaluate the impact of small molecules on the cytotoxic activity of effector cells such as NK cells.

The cytopathic effects associated to measles virus infection has finally been measured by NanoLuc quantification in supernatants of Twlnne cells (FIG. 5). These results show that the NanoLuc enzyme is released and detectable in the culture supernatants at day 3 in infected cultures. It is therefore possible to quantify precisely the cytolytic efficiency of viruses thanks to the method of the invention.

### BIBLIOGRAPHIC REFERENCES

Corey et al. (1997) J. Immunol. Meth. 207:43-51
Coutant E P & Janin Y L, Chem. Eur. J., 2015
Hall M P et al, ACS Chem Biol 2012, 7, 1848-1857
Karimi M A et al, PLOS ONE 2014
Lee et al. Biochem. Biophys. Res. Commun. 2014
Nakagawa Y. et al, Biomed. Res. 2011
Naldini & al, Science. 1996 Apr. 12; 272(5259):263-7
Wahlberg B J. Et al, J. immunol. Methods 2001

1. An in vitro method for testing the cytotoxicity of a candidate compound for living target cells, said method comprising at least the following steps:
a) contacting living target cells that constitutively express in their cytosol a luminescent enzyme with said candidate compound, said luminescent enzyme being stable in biological media including cellular lysates, and having a size comprised between 10 and 30 kDa,
b) isolating the supernatant of the coculture of step a) in a second recipient,
c) adding in said second recipient the substrate of said luminescent enzyme, and
d) measuring the luminescence emitted in said second recipient,
wherein the cytotoxicity of said candidate compound is proportional to the increase in luminescence measured in step d).

2. The method of claim 1, wherein the luminescence of said luminescent enzyme is at least five time brighter than that of the native Renilla luciferase.

3. The method of claim 1 or 2, wherein said luminescent enzyme is the NanoLuc® enzyme derived of Oplophorus gracilirostris.

4. The method of any one of claims 1 to 3, wherein said candidate compound is chosen in the group consisting of: a chemical compound, a therapeutic compound, a dermatologic compound, a chemical detergent, an effector cell, a virus, a bacteria, a lytic protein or a lytic protein complex.

5. The method of claim 4 wherein said effector cell is a population of Natural Killer cells, of T CD8+ lymphocytes, of monocytes, of macrophages, of dendritic cells, of virus-infected cell or tumoral cell.

6. The method of any one of claims 1 to 5, wherein the luminescence of said luminescent enzyme is measured in step d) by a luminometer.

7. The method of any one of claims 1 to 6, wherein steps a) to d) are performed automatically.

8. The method of any one of claims 4 to 7, wherein the ratio between said effector cells and said living target cells is comprised between about 0.1:1 to about 10:1, preferably between about 0.5:1 and 5:1.

9. The method of any one of claims 1 to 8, wherein said target living cells are chosen in the group consisting of: bacteria, archea and eukaryotic cell lines, and are preferably tumoral eukaryotic cell lines.

10. The method of any one of claims 1 to 9, wherein said living target cells have been transduced by a lentivirus or a retrovirus expressing the gene coding for said luminescent enzyme under a constitutive promoter.

11. The method of any one of claims 1 to 10, wherein the measuring step d) occurs no later than 4 hour after the living target cells have been contacted by the candidate compound.

12. An automated in vitro method for screening concomitantly the cytotoxicity of several candidate compounds, said method repeating the steps as defined in claims 1 to 11 for each candidate compound concomitantly.

13. An in vitro method for detecting the immunodulatory efficiency of a candidate compound on effector cells, comprising the following steps:
a) Performing the method as defined in any one of claims 4 to 11 in the absence of said candidate compound,
b) Performing the method as defined in any one of claims 4 to 11 in the presence of said candidate compound,
c) Concluding that the candidate compound has an immunosuppressive activity if the cytotoxicity measured in step b) is lower than the cytotoxicity measured in step a),
d) concluding that the candidate compound has an immunostimulatory activity if the cytotoxicity measured in step b) is higher than the cytotoxicity measured in step a).

14. The method of claim 13, wherein said effector cells are NK cells, cytotoxic T lymphocytes, monocytes, macrophages, dendritic cells, virus-infected cells or tumoral cells.

15. The method of claim 13 or 14, wherein the concluding step c) or d) occurs no later than 4 hours after the living target cells have been first contacted by said effector cells.
